(19) United States
(12) Patent Application Publication
(10) Pub. No.: US 2010/0292728 A1
(43) Pub. Date: Nov. 18, 2010

(54) JAW-TYPE GRAB FOR A SURGICAL INSTRUMENT

(76) Inventor: Peter METZGER, Wien (AT)

Correspondence Address: KF ROSS PC, 5683 RIVERDALE AVENUE, SUITE 203 BOX 900, BRONX, NY, 10471-0900, US

(21) Appl. No.: 12/779,297

(22) Filed: May 13, 2010

(30) Foreign Application Priority Data
May 14, 2009 (AT) A745/2009

(51) Int. Cl. A61B 17/28

(52) U.S. Cl. 606/206

(57) ABSTRACT

A grab for a surgical instrument has an elongated tube having a rigid outer part and a rigid inner part interconnected by a pivot pin extending along an axis transverse to the parts. A pair of jaws are pivotal on an outer end of the outer part between open and closed positions. An elongated actuating rod has a rigid outer part in the outer tube part and connected to the jaws, a rigid inner part in the inner tube part, and a flexible center part longitudinally interconnecting the rod inner and outer parts and passing through or around the pivot pin. The pivot pin is formed on the pivot axis with a throughgoing slot and the flexible part of the rod extends through the slot.

### FIELD OF THE INVENTION

The present invention relates to a jaw-type grab. More particularly this invention concerns such a grab used for surgery, in particular for laparoscopic or minimally invasive surgery.

### BACKGROUND OF THE INVENTION

A jaw-type grab is typically mounted on an outer end of a shaft for pivoting about an axis that is perpendicular to a longitudinal axis of the head. To this end a pivot pin is provided on the shaft that defines this pivot axis and that subdivides the instrument into an outer part or head having a pair of openable and closable jaws and an inner part that is held by the operator. A rod extending along the shaft is coupled to the jaws so that longitudinal shifting of the shaft in one direction, typically outward, opens or spreads the jaws and opposite movement closes them. Such a grab is commonly used in minimally invasive surgery, also known as keyhole surgery, buttonhole surgery, laparoscopy in the abdominal region, thorascopy in the thorax. This is surgery with the smallest possible trauma, in that the surgeon inserts and guides the surgical instrument into the body and to the target through a narrow incision and/or a natural body orifice.

The advantages of the minimally invasive surgery arise, on the one hand, from the much smaller incisions which leave the abdominal wall or the thorax largely intact, and on the other hand, with this surgery technique, no physical load (tension, pressure, etc.) for abdominal wall, peritoneum, and adjacent organs due to the use of spreader hooks, absorption cloths, spatulas etc. occurs. Therefore, the patient has less pain after the surgery and needs less or no pain medications and is able to breathe, cough, and get up normally shortly after the surgery.

From a business point of view, higher surgery costs (instruments, technology such as video camera and monitor, is apparatuses for gas insufflation etc.), in some cases more complicated anaesthesia and, in the initial phase, longer operation times are to be considered. This is more than compensated for by the short; however, in contrast to that, the duration of hospitalization and the complication rate decrease.

The use in minimally invasive surgery places very high demands on surgical instruments with respect to their functionality and handling. On the one hand, they must have some rigidity, but, on the other hand, they should when necessary be flexible and elastic to perform a surgery in a problem-free and fast manner.

All conventional surgical jaw-type grabs, due to their complicated handling and poor gripping flexibility, are only suitable to a limited extent for precise surgical actions because they can only rotate, and their gripping jaws can open and close only longitudinally.

### OBJECTS OF THE INVENTION

It is therefore an object of the present invention to provide an improved jaw-type grab for a surgical instrument.

Another object is the provision of such an improved jaw-type grab for a surgical instrument that overcomes the above-given disadvantages, in particular that is whose jaws open and close transversely and that stay closed or opened even if the head is pivoted.

### SUMMARY OF THE INVENTION

A grab for a surgical instrument has according to the invention an elongated tube having a rigid outer part and a rigid inner part interconnected by a pivot pin extending along an axis transverse to the parts. A pair of jaws are pivotal on an outer end of the outer part between open and closed positions. An elongated actuating rod has a rigid outer part in the outer tube part and connected to the jaws, a rigid inner part in the inner tube part, and a flexible center part longitudinally interconnecting the rod inner and outer parts and passing through or around the pivot pin.

Preferably according to the invention the pivot pin is formed on the pivot axis with a throughgoing slot and the flexible part of the rod extends through the slot.

Thus the jaw-type grab according to the invention allows the head to pivot without the jaws opening or closing.

In an advantageous embodiment of the invention, the elastic deflecting element is guided in a diametrally throughgoing slot in the pivot pin.

### BRIEF DESCRIPTION OF THE DRAWING

The above and other objects, features, and advantages will become more readily apparent from the following description, reference being made to the accompanying drawing in which:

FIG. 1 is a perspective view of the grab with the shaft straight and the jaws open;

FIGS. 2 and 3 are large-scale views of the grab head with the jaws open and closed and the shaft flexed; and

FIG. 4 is a large-scale view of a structural detail of the invention.

### SPECIFIC DESCRIPTION

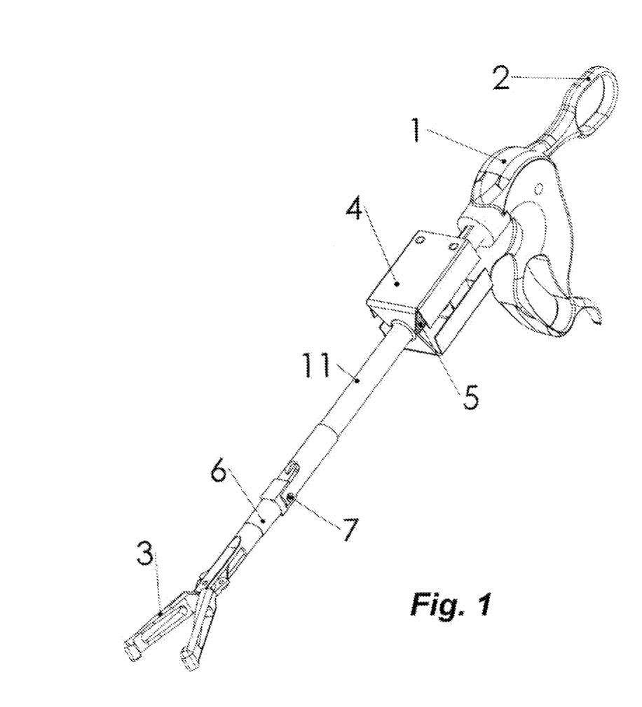

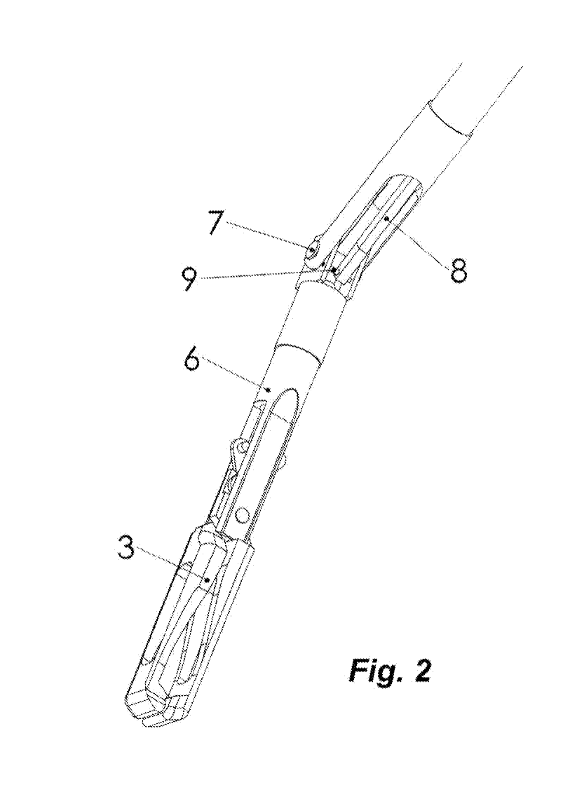

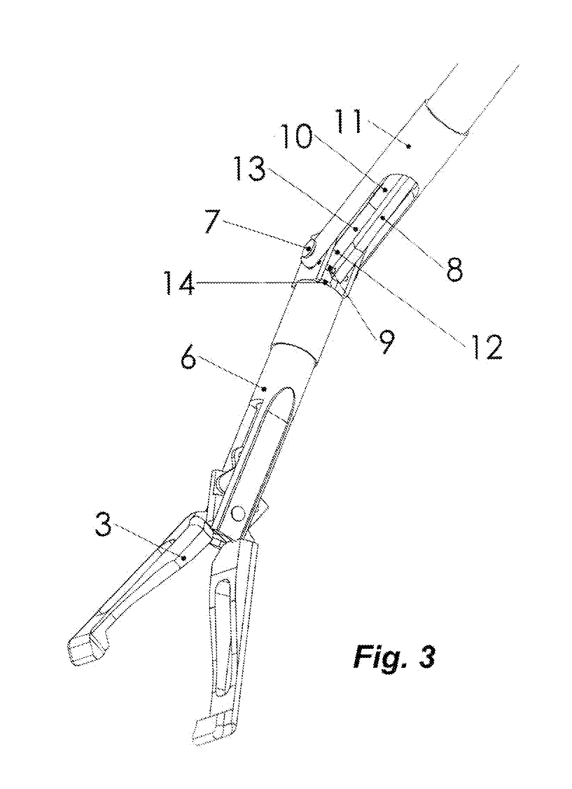

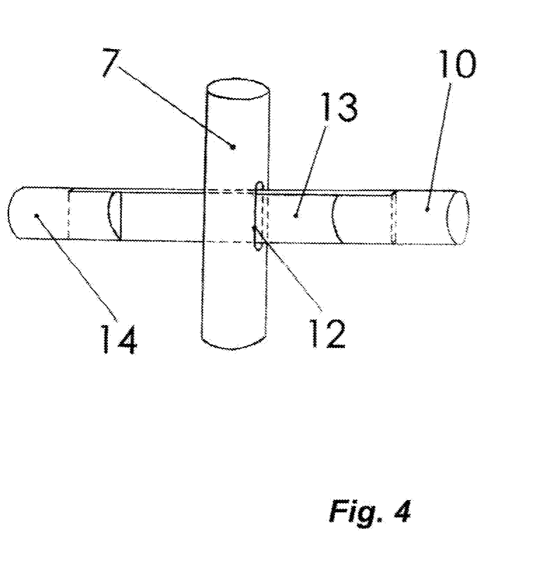

As seen in FIG. 1 a surgical jaw-type grab according to the invention according to the invention has a handle 1 with an actuating lever 2 for opening and closing two pivotal jaws 3. Another actuating element 4 is shiftable against the force of a spring 5 to pivot a head tube 6 carrying the jaws 4 on the other end of a rear shaft tube 11. The head tube 6 pivots about a transverse axis A defined by a pivot pin 7. FIGS. 1 and 3 show the jaws 3 open or spread and FIG. 2 shows them closed, and FIGS. 2 and 3 show the head tube 6 pivoted at an angle to the tube 11 while FIG. 1 shows the head tube 6 coaxial with the rear tube 11.

A push rod 8 extends coaxially through the rear tube 11 and has a front end pivoted offset from the pin 7 at 9 on the rear end of the head tube 6 so that axial movement of the rod 8 will pivot the head 6 about the pin 9. The rear end of the rod 8 is attached to the element 4 so that axially shifting the element 4 pivots the head tube 6.

The jaws 3 are pivoted between their open and closed positions by an actuating rod assembly comprised of a rigid rear rod 10 extending coaxially in the rear tube adjacent the rod 8, a rigid front rod 14 extending coaxially through the head tube 6 and having a front end pivoted on rear ends of the pivotal jaws 3, and a flexible flat bar 13 fixed between the front end of the rod 10 and the rear end of the rod 14. The pivot pin 7 is formed with a diametrally throughgoing slot 12 through which the flexible part 13 passes. The rear end of the rod 10 is connected to the pivotal handle 2 so that pivoting this handle 2 control opening and closing of the jaws 3.

Since force is transmitted at the axis A where the device can flex, this angle can be changed without appreciably affecting the positions of the jaws 3. Thus it is possible to adjust angular position of the head tube and the position of the jaws 3 independently of each other.

Within the context of the invention, numerous modifications are possible. In particular, the invention is not limited to surgical instruments. On the contrary, its use can be implemented in all cases in which actions can be carried out only through a small opening. Also, diagnostic or therapeutic actions should be mentioned here.

1. A grab for a surgical instrument, the grab comprising:
an elongated tube having an outer part and an inner part;
a pivot pin interconnecting the parts and extending along an axis transverse to the parts, whereby the tube parts can pivot relative to each other about the axis;
a pair of jaws pivotal on an outer end of the outer part between open and closed positions;
an elongated actuating rod having an outer part in the outer tube part and connected to the jaws, an inner part in the inner tube part, and a flexible center part longitudinally interconnecting the rod inner and outer parts; and
means on the inner part for shifting the outer rod part and thereby shifting the rod inner part and pivoting the jaws.

2. The surgical-instrument grab defined in claim 1 wherein the pivot pin is formed on the pivot axis with a throughgoing slot, the flexible part of the rod extending through the slot.

3. The surgical-instrument grab defined in claim 2 wherein the slot is elongated parallel to the axis and the flexible part of the rod extends diametrally through the pin in the slot.
